(19) United States
(12) Patent Application Publication
(10) Pub. No.: US 2024/0252769 A1
(43) Pub. Date: Aug. 1, 2024

(54) System to Detect Markers of a Lipohypertrophy Lesion

(71) Applicant: Becton, Dickinson and Company, Franklin Lakes, NJ (US)

(72) Inventors: John Adams, Killester (IE); David MacManus, Dublin 4 (IE); Steve Beguin, Nenzlingen (CH)

(21) Appl. No.: 18/578,905

(22) PCT Filed: Jul. 13, 2022
(86) PCT No.: PCT/US22/36941

§ 371 Date: Jan. 12, 2024

Related U.S. Application Data
(60) Provisional application No. 63/221,072, filed on Jul. 13, 2021.

(51) Int. Cl. A61M 5/42; G16H 20/17

(52) U.S. Cl. CPC A61M 5/427; G16H 20/17; A61M 2205/3303; A61M 2205/3306; A61M 2210/04; A61M 2230/50

(57) ABSTRACT

A system for detecting a marker of abnormal skin tissue to assess the suitability of a site for subcutaneous injection of a drug, includes a housing, one or more sensors received by the housing, the one or more sensors configured to detect a marker of abnormal skin tissue susceptible to impair the absorption dynamics of the drug to be injected, one or more indicators configured to provide an indication from the one or more sensors whether a marker of abnormal skin tissue has been detected, and one or more computing devices in communication with the one or more sensors and the one or more indicators.

### CROSS-REFERENCE TO RELATED APPLICATION

The present application claims priority to U.S. Provisional Application Ser. No. 63/221,072, entitled “System to Detect Markers of a Lipohypertrophy Lesion”, filed Jul. 13, 2021, the entire disclosure of which is hereby incorporated by reference in its' entirety.

### BACKGROUND OF THE INVENTION

### Field of the Disclosure

The present application relates generally to a system for detecting markers of a skin tissue abnormality, such as a lipohypertrophy lesion.

### Description of the Related Art

Lipohypertrophy is a condition where adipocytes accumulate large quantities of lipids in their liposomes and become hypertrophied. This condition results in a modification of the adipose tissue with highly enlarged cells and denser extracellular matrix content, leading to lower vascularization of the tissue. This condition is induced by repeatedly exposing adipocytes to insulin, and is common in a patient receiving insulin therapy. This condition is localized and can be avoided if the injection sites are rotated. Further, the lower vascularization of the lipohypertrophic lesions, combined with the denser extracellular matrix and the impaired signaling pathways of the hypertrophied adipocytes affect the pharmacokinetics of the drug and lower its efficacy, i.e., delayed release into the blood stream, thereby leading to poor management of the medical condition of the patient. Therefore, there is a need to identify whether or not a specific location or injection site includes a lipohypertrophic lesion and if the site is suitable for the injection of insulin.

Other types of skin abnormalities impeding the absorption rate or the pharmacokinetics of a drug injected in subcutaneous tissue may be any of scar tissue, damaged tissue, bruised tissue, inflamed tissue, cyst, mole, thick dermis, thin subcutaneous tissue, tumoral tissue, abnormal extracellular matrix composition (e.g. collagen, hyaluronan, elastin), fibrous tissue, or abnormal cellular morphology or arrangement.

### SUMMARY OF THE INVENTION

In one aspect or embodiment, a system for detecting a marker of abnormal skin tissue to assess the suitability of a site for subcutaneous injection of a drug, includes a housing, one or more sensors received by the housing, the one or more sensors configured to detect a marker of abnormal skin tissue susceptible to impair the absorption dynamics of the drug to be injected, one or more indicators configured to provide an indication from the one or more sensors whether a marker of abnormal skin tissue has been detected, and one or more computing devices in communication with the one or more sensors and the one or more indicators.

The housing may be formed integrally with a drug delivery device or may be configured to be secured to a drug delivery device.

The abnormal skin tissue may be a lipohypertrophy lesion.

The one or more sensors may be configured to determine at least one of temperature, temperature conductivity, vascularization, oxygen level, carbon dioxide level, cell size, extracellular matrix composition, impedance, capacitance, and mechanical properties of an injection site.

The one or more sensors may include at least one of a temperature sensor, a thermal conductivity sensor, a heating element, a capacitive sensor, an electrode for impedance tomography, a pulse oximetry sensor, a photoplethysmography sensor, a strain gauge, and an ultrasonic imaging sensor.

The one or more indicators may include at least one of a visual indicator and an audible indicator.

The system may further include an accelerometer configured to determine a position or movement of the one or more sensors relative to a skin surface of a patient. The one or more sensors may be configured to directly contact a skin surface of a patient. The one or more sensors may be configured to be spaced from a skin surface of a patient during use of the system. The system may further include a mobile device configured to be in communication with the one or more sensors. The one or more indicators may include a plurality of secondary indicators positioned on the housing and configured to indicate a direction to move the system.

In a further aspect or embodiment, a method for detecting markers of abnormal skin tissue at an injection site for a drug delivery device, includes: scanning a skin surface of a patient within one or more sensors configured to detect a marker of abnormal skin tissue; measuring a value associated with the one or more sensors using a computing device; and providing an indication to a patient whether the injection site includes markers of abnormal skin tissue using one or more indicators.

The abnormal skin tissue may include at least one of a lipohypertrophy lesion, a cyst, a mole, some scar tissue, a tumor, compact skin tissue, dense extracellular matrix, and abnormal cell aggregate.

The method may further include scanning the skin surface of a patient including moving the drug delivery device along the skin surface of the patient. The method may further include: recording an image of the skin surface of the patient using a thermographic camera; and providing an indication to the patient where the skin surface of the patient is free from markers of a lipohypertrophy lesion. The thermographic camera may be in communication with a mobile device, with the mobile device utilized to provide the indication to the patient where the skin surface of the patient is free from markers of a lipohypertrophy lesion. The method may include attaching a housing including the one or more sensors to a drug delivery device.

The method may include: recording a plurality of positions of the one or more sensors using an accelerometer; recording a plurality of values measured by the one or more sensors at the corresponding position of the one or more sensors; and mapping a portion of the skin surface of the patient with the plurality values measured by the one or more sensors in conjunction with the positions calculated from the data measured with the accelerometer.

### BRIEF DESCRIPTION OF THE DRAWINGS

The above-mentioned and other features and advantages of this disclosure, and the manner of attaining them, will become more apparent and the disclosure itself will be better understood by reference to the following descriptions of aspects of the disclosure taken in conjunction with the accompanying drawings, wherein:

FIG. 1 is a perspective view of a conventional drug delivery device.

FIG. 2 is a schematic view of a system for detecting markers of a lipohypertrophy lesion according to one aspect or embodiment of the present application, showing the system integrated with a drug delivery device.

FIG. 3 is a schematic view of a system for detecting markers of a lipohypertrophy lesion according to a further aspect or embodiment of the present application, showing the system connected to a drug delivery device.

FIG. 4 is a perspective view of the system of FIG. 3.

FIG. 5 is a sensor output image according to one aspect or embodiment of the present application.

FIG. 6 is a composite sensor output image according to one aspect or embodiment of the present application.

FIG. 7 is a schematic view of an electronic subsystem according to one aspect or embodiment of the present application.

Corresponding reference characters indicate corresponding parts throughout the several views. The exemplifications set out herein illustrate exemplary aspects of the disclosure, and such exemplifications are not to be construed as limiting the scope of the disclosure in any manner.

### DETAILED DESCRIPTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting. All numbers and ranges used in the specification and claims are to be understood as being modified in all instances by the term “about”. By “about” is meant plus or minus twenty-five percent of the stated value, such as plus or minus ten percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. A computing device may also be a desktop computer or other form of non-mobile computer. In non-limiting embodiments, a computing device may include a GPU. In non-limiting embodiments, a computing device may be comprised of a plurality of circuits.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments, a message may refer to a network packet (e.g., a data packet and/or the like) that includes data. It will be appreciated that numerous other arrangements are possible.

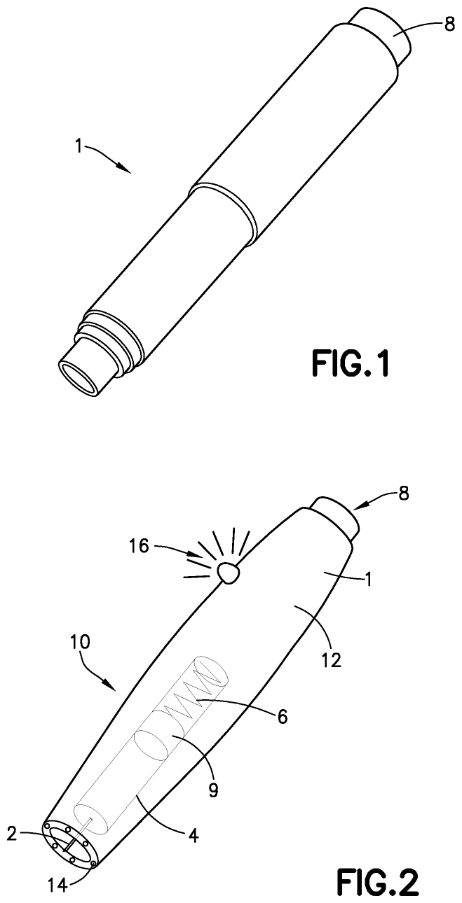

Referring to FIGS. 1 and 2, a conventional drug delivery device 1 is shown. The drug delivery device 1 includes a needle 2, a drug reservoir 4 in fluid communication with the needle 2, and a power source 6 for expelling or delivering fluid from the drug reservoir 4 to the needle 2. The drug delivery device 1 includes a button 8 utilized to actuate the drug delivery device 1. Upon actuation, the needle 2 pierces a skin surface of a patient and the power source 6 delivers a dose of medication from the drug reservoir 4 into the patient via the needle 2. The drug delivery device 1 may be an auto-injector and include a needle shield, with the needle shield configured to retract to allow insertion of the needle 2 into a patient and subsequently extend after completing an injection to shield the needle 2. The power source 6 may be a spring, electric motor, chemical reaction, electrochemical reaction, pneumatic system, and/or hydraulic system, or a mechanical movement resulting from any combination thereof. The power source 6 may move a component into engagement with a stopper 9 within the drug reservoir 4. The drug delivery device 1 may also be a pen injector, a wearable injector, or other suitable drug delivery device.

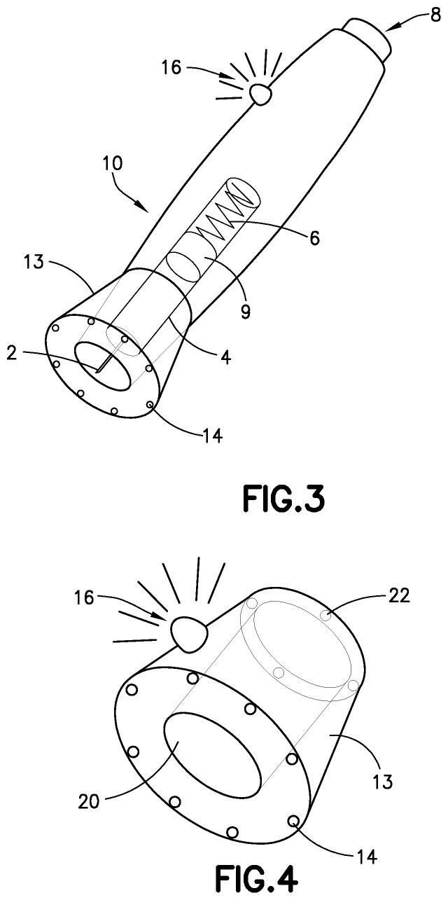

Referring to FIGS. 2, 3, and 7, according to one aspect or embodiment of the present application, a system 10 for detecting markers of a lipohypertrophy lesion includes a housing 12 formed integrally with the drug delivery device 1 (FIG. 2) or a housing 13 configured to be secured to the drug delivery device 1 (FIG. 3), one or more sensors 14 received by the housing 13 with the one or more sensors 14 configured to detect a marker of a lipohypertrophy lesion, one or more indicators 16 configured to provide an indication from the one or more sensors 14 whether a marker of a lipohypertrophy lesion has been detected, and one or more computing devices 18 in communication with the one or more sensors 14 and the one or more indicators 16. The system 10 may be utilized with the drug delivery device 1 shown in FIG. 1 and described above, although the system 10 may be utilized in connection with any suitable drug delivery device or on its own.

Referring to FIGS. 3 and 4, the system 10 may be secured to the drug delivery device 1 via an interference or friction fit, a clamp, adhesive, or any other suitable arrangement. The housing 13 of the system 10 defines an opening 20 and is positioned at a distal end of the drug delivery device 1, with the needle 2 of the drug delivery device 1 configured to extend through the opening 20 of the housing 13. In a further aspect or embodiment, the system 10 shown in FIG. 4 may be utilized as a standalone device separated from the drug delivery device 1 and prior to use of the drug delivery device 1.

In one aspect or embodiment, the one or more sensors 14 are configured to determine at least one of temperature, temperature conductivity, vascularization, oxygen level, carbon dioxide level, cell size, extracellular matrix composition, impedance, capacitance, water content, lipid content, abundance of lymphatic vessels, and mechanical properties of an injection site. A lipohypertrophied lesion will have different physical and/or physiological differences compared to normal tissue or tissue not having a lipohypertrophied lesion. Measuring these physical and/or physiological differences using one or more of the properties via the one or more sensors 14 allows a lipohypertrophied lesion to be detected.

In one aspect or embodiment, the one or more sensors 14 include at least one of a temperature sensor, a thermal conductivity sensor, a heating element, a capacitive sensor, an electrode for impedance tomography, a pulse oximetry sensor, a strain gauge, and an ultrasonic imaging sensor. The temperature sensor is configured to detect differences in skin temperatures between different portions of the skin surface of the patient. The one or more sensors 14 may include a heating element and measure thermal conductivity of the skin to detect a lipohypertrophic lesion. The pulse oximetry sensor may be configured to use photoplethysmophraghy to measure the blood flow to detect the presence of a lipohypertrophic lesion. The pulse oximetry sensor may be configured to measure oxygen and carbon dioxide levels to detect a lipohypertrophic lesion. The lower vascularization in lipohypertrophic lesions is expected to display biomarkers such as lower oxygen levels, lower topical temperature, and difference of interstitial fluid content or composition. The denser and more lipid-rich tissue of a lipohypertrophic lesion may display biomarkers such as lower thermal conductivity, lower thermal inertia, or different capacitance or impedance properties compared to normal tissue. The strain gauge may be configured to measure the viscoelastic properties of the tissue, including the young modulus, stiffness, and/or damping of the tissue in the area being measured by micro-indentation, nano-indentation, and/or dynamic indentation. The mechanical properties of the tissue may also be measured using ultrasounds or ultrasonic imaging. The difference in water and/or lipid content of the tissue may be measured by impedance measurement, impedance spectroscopy, and/or by measuring the capacitance of the tissue.

Referring to FIGS. 2-4, the one or more indicators 16 may include at least one of a visual indicator and an audible indicator. As shown in FIGS. 2 and 3, the indicator 16 may be provided on the drug delivery device 1 itself. As shown in FIG. 4, the one or more indicators 16 may be provided on the housing 13, which is separate from the drug delivery device 1 and connected to the drug delivery device 1, and may also include a plurality of secondary indicators 22 which may be positioned circumferentially around the housing 13. The plurality of secondary indicators 22 may be configured to show a direction to move the drug delivery device 1, e.g., up, down, left, and right, and/or to indicate where a suitable injection site has been identified.

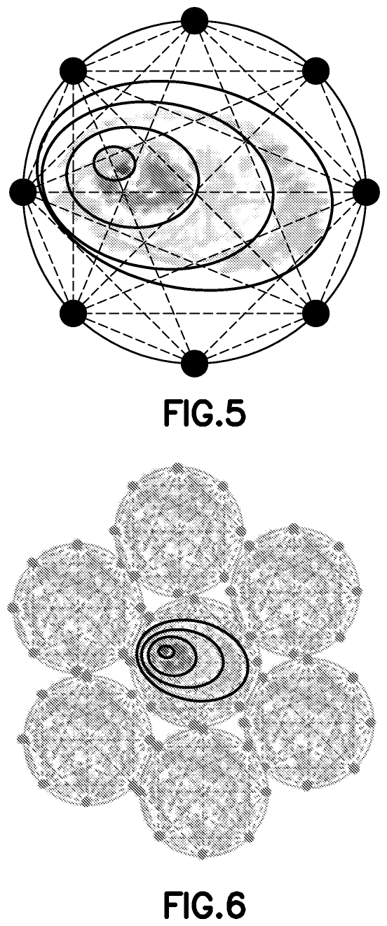

Referring to FIGS. 5 and 6, in one aspect or embodiment, the system 10 further includes an accelerometer (not shown) configured to determine a position of the one or more sensors 14 relative to a skin surface of a patient. The one or more sensors 14 can be coupled with the accelerometer to record values and map a surface, as shown in FIGS. 5 and 6, larger than the area covered by the one or more sensors 14. More specifically, FIG. 5 shows a single sensor capture in an area surrounded by the one or more sensors 14. FIG. 6 shows a mapped skin area using an accelerometer to stitch individual measurements or captures, such as the capture shown in FIG. 5. The one or more sensors 14 and/or accelerometer can be coupled with an algorithm on the computing device to determine if the position of the drug delivery device 1 is above, below, right, or left of a suitable injection site, i.e., an injection site free from lipohypertrophic lesions.

In one aspect or embodiment, the one or more sensors 14 are configured to directly contact a skin surface of a patient. In a further aspect or embodiment, the one or more sensors 14 are configured to be spaced from a skin surface of a patient during use of the system 10.

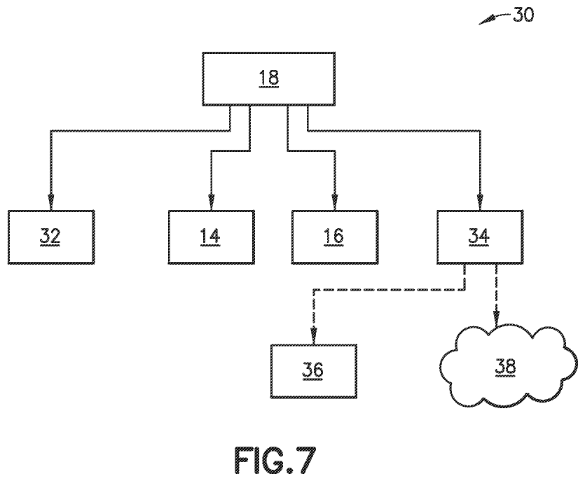

Referring to FIG. 7, in one aspect or embodiment, the system 10 includes an electronic subsystem 30 including the computing device 18, which is in communication with an energy source 32, the one or more sensors 14, the one or more indicators 16, and a communication device 34. The communication device 34 may further be in communication with a mobile device 36. The communication device 34 may also be in communication with an external system 38, such as an external server, internet, network, etc. The energy source 32 may be a battery, although other suitable energy sources may be utilized.

In a further aspect or embodiment, a method for detecting markers of a lipohypertrophy lesion at an injection site for the drug delivery device 1, includes: scanning a skin surface of a patient within the one or more sensors 14; measuring a value associated with the one or more sensors 14 using the computing device 18; and providing an indication to a patient whether the injection site includes markers of a lipohypertrophy lesion using one or more indicators 16. Scanning the skin surface of the patient may include moving the drug delivery device 1 along the skin surface of the patient. In one aspect or embodiment, the method includes recording an image of the skin surface of the patient using a thermographic camera; and providing an indication to the patient where the skin surface of the patient is free from markers of a lipohypertrophy lesion. The thermographic camera may be in communication with the mobile device 36, with the mobile device 36 utilized to provide the indication to the patient where the skin surface of the patient is free from markers of a lipohypertrophy lesion. In one aspect or embodiment, the system 10 is configured to communicate and cooperate with an application running on the mobile device 36. The method may include attaching the housing 13, including the one or more sensors 14, to the drug delivery device 1.

In one aspect or embodiment, the method includes: recording a plurality of positions of the one or more sensors 14 using the accelerometer; recording a plurality of values measured by the one or more sensors 14 at the corresponding position of the one or more sensors 14; and mapping a portion of the skin surface of the patient with the plurality values measured by the one or more sensors 14.

The system 10 may perform one or more processes described herein. The system 10 may perform these processes based on a processor executing software instructions stored by a computer-readable medium, such as memory and/or storage component. A computer-readable medium may include any non-transitory memory device. A memory device includes memory space located inside of a single physical storage device or memory space spread across multiple physical storage devices. Software instructions may be read into memory and/or storage from another computer-readable medium or from another device via a communication interface. When executed, software instructions stored in memory and/or storage component may cause a processor to perform one or more processes described herein. Additionally, or alternatively, hardwired circuitry may be used in place of or in combination with software instructions to perform one or more processes described herein. Thus, embodiments described herein are not limited to any specific combination of hardware circuitry and software.

While this disclosure has been described as having exemplary designs, the present disclosure can be further modified within the spirit and scope of this disclosure. This application is therefore intended to cover any variations, uses, or adaptations of the disclosure using its general principles. To the extent possible, one or more features of any embodiment or aspect can be combined with one or more features of any other embodiment or aspect. Further, this application is intended to cover such departures from the present disclosure as come within known or customary practice in the art to which this disclosure pertains and which fall within the limits of the appended claims.

1. A system for detecting a marker of abnormal skin tissue to assess the suitability of a site for subcutaneous injection of a drug, the system comprising:
a housing;
one or more sensors received by the housing, the one or more sensors configured to detect a marker of abnormal skin tissue susceptible to impair the absorption dynamics of the drug to be injected;
one or more indicators configured to provide an indication from the one or more sensors whether a marker of abnormal skin tissue has been detected; and
one or more computing devices in communication with the one or more sensors and the one or more indicators.

2. The system of claim 1, wherein the housing is formed integrally with a drug delivery device or configured to be secured to a drug delivery device.

3. The system of claim 1, wherein the abnormal skin tissue is a lipohypertrophy lesion.

4. The system of claim 1, wherein the one or more sensors are configured to determine at least one of temperature, temperature conductivity, vascularization, oxygen level, carbon dioxide level, cell size, extracellular matrix composition, impedance, capacitance, and mechanical properties of an injection site.

5. The system of claim 1, wherein the one or more sensors comprises at least one of a temperature sensor, a thermal conductivity sensor, a heating element, a capacitive sensor, an electrode for impedance tomography, a pulse oximetry sensor, a photoplethysmography sensor, a strain gauge, and an ultrasonic imaging sensor.

6. The system of claim 1, wherein the one or more indicators comprises at least one of a visual indicator and an audible indicator.

7. The system of claim 1, further comprising an accelerometer configured to determine a position of the one or more sensors relative to a skin surface of a patient.

8. The system of claim 1, wherein the one or more sensors are configured to directly contact a skin surface of a patient.

9. The system of claim 1, wherein the one or more sensors are configured to be spaced from a skin surface of a patient during use of the system.

10. The system of claim 1, further comprising a mobile device configured to be in communication with the one or more sensors.

11. The system of claim 1, wherein the one or more indicators comprises a plurality of secondary indicators positioned on the housing and configured to indicate a direction to move the system.

12. A method for detecting markers of abnormal skin tissue at an injection site for a drug delivery device, the method comprising:
scanning a skin surface of a patient within one or more sensors configured to detect a marker of abnormal skin tissue;
measuring a value associated with the one or more sensors using a computing device; and
providing an indication to a patient whether the injection site includes markers of abnormal skin tissue using one or more indicators.

13. The method of claim 12, wherein the abnormal skin tissue comprises at least one of a lipohypertrophy lesion, a cyst, a mole, some scar tissue, a tumor, compact skin tissue, dense extracellular matrix, and abnormal cell aggregate.

14. The method of claim 12, wherein scanning the skin surface of a patient comprises moving the drug delivery device along the skin surface of the patient.

15. The method of claim 12, wherein the method further comprises:
recording an image of the skin surface of the patient using a thermographic camera; and
providing an indication to the patient where the skin surface of the patient is free from markers of a lipohypertrophy lesion.

16. The method of claim 15, wherein the thermographic camera is in communication with a mobile device, and wherein the mobile device is utilized to provide the indication to the patient where the skin surface of the patient is free from markers of a lipohypertrophy lesion.

17. The method of claim 12, wherein the method further comprises:
attaching a housing including the one or more sensors to a drug delivery device.

18. The method of claim 12, wherein the method comprises:
recording a plurality of positions of the one or more sensors using an accelerometer;
recording a plurality of values measured by the one or more sensors at the corresponding position of the one or more sensors; and
mapping a portion of the skin surface of the patient with the plurality values measured by the one or more sensors in conjunction with the positions calculated from data measured with the accelerometer.
